(19) United States
(12) Patent Application Publication
(10) Pub. No.: US 2023/0404661 A1
(43) Pub. Date: Dec. 21, 2023

(54) CATHETER

(71) Applicant: Japan Lifeline Co., Ltd., Tokyo (JP)

(72) Inventor: Kohei SAKAKI, Tokyo (JP)

(21) Appl. No.: 18/447,933

(22) Filed: Aug. 10, 2023

Related U.S. Application Data
(63) Continuation of application No. PCT/JP2021/024865, filed on Jun. 30, 2021.

(51) Int. Cl. A61B 18/14

(52) U.S. Cl. CPC A61B 18/1492; A61B 2018/00267

(57) ABSTRACT

A catheter according to an embodiment of the disclosure includes a catheter shaft extending in an axial direction and having a near-distal end structure including a plurality of electrodes, and a handle mounted on a proximal end side of the catheter shaft. The handle includes a handle body extending in the axial direction, and an operation mechanism configured to be rotatable with respect to the handle body about a rotation axis extending along the axial direction. The operation mechanism is rotationally operated when the near-distal end structure is rotationally operated about the rotation axis while a length of the near-distal end structure in the axial direction is fixed.

### CROSS REFERENCE TO RELATED APPLICATIONS

This application is a continuation application of International Application No. PCT/JP2021/024865, filed on Jun. 30, 2021, the entire contents of which are hereby incorporated by reference.

### TECHNICAL FIELD

The disclosure relates to a catheter including a catheter shaft.

### BACKGROUND

A catheter (electrode catheter) including an electrode provided at a catheter shaft (for example, JP 3830521 B) is an example of a medical device including an electrode near its distal end. In the catheter in JP 3830521 B, the structure near the distal end of the catheter shaft is configured to be deformable.

### SUMMARY

In a catheter such as that described above, generally requires improved convenience. It is desirable to provide a catheter capable of improving convenience.

A catheter according to an embodiment of the disclosure includes a catheter shaft extending in an axial direction and having a near-distal end structure including a plurality of electrodes, and a handle mounted on a proximal end side of the catheter shaft. The handle includes a handle body extending in the axial direction, and an operation mechanism configured to be rotatable with respect to the handle body about a rotation axis extending in the axial direction, the operation mechanism being rotationally operated when the near-distal end structure is rotationally operated about the rotation axis while a length of the near-distal end structure in the axial direction is fixed.

The catheter according to an embodiment of the disclosure includes the operation mechanism that is configured to be rotatable with respect to the handle body about the rotation axis and is rotationally operated when the near-distal end structure is rotationally operated. With this configuration, since the near-distal end structure rotates about the rotation axis in response to the rotation operation, the position of the electrode in the near-distal end structure can be freely adjusted in the radial direction or the circumferential direction about the axial direction while the length of the near-distal end structure in the axial direction is fixed.

In the catheter according to an embodiment of the disclosure, the operation mechanism may be further configured to be slidable in the axial direction in the handle body and be slid in the axial direction during a deformation action in which a shape of the near-distal end structure is changed between a first shape and a second shape. The first shape may be a non-deployed shape in which the near-distal end structure is not deployed in the axial direction, and the second shape may be a deployed shape in which the near-distal end structure is deployed from the non-deployed shape in the axial direction. In such a case, during the deformation action, in a state where the operator holds the handle body with one hand, the operator can perform an operation on the operation mechanism with that hand (the same hand). That is, for example, an operation using both hands of the operator as in the case of an operation of pushing a wire (operation wire) used for the slide operation into the handle body with the other hand is not required, and the operation (slide operation) during the deformation action can be easily performed using only one hand of the operator. In addition, since the position in the radial direction of the electrode in the near-distal end structure can also be adjusted in accordance with the deformation action, for example, the position of the electrode can also be adjusted in accordance with the thickness (size of the diameter) of the blood vessel of the patient. Accordingly, the convenience when using the catheter can be further improved.

In this case, the near-distal end structure may be settable to any intermediate shape between the non-deployed shape and the deployed shape according to a slide position of the operation mechanism in the handle body. In this case, the near-distal end structure can be set to any intermediate shape, and thus the convenience can be further improved.

A distal end side of an operation wire used in the rotation action and the deformation action may be fixed to the near-distal end structure, and a proximal end side of the operation wire may be fixed to the operation mechanism in the handle body via a gear configured to rotate in conjunction with the rotation operation. In this case, since the rotation action and the deformation action can be easily performed, convenience when using the catheter is further improved.

The near-distal end structure may include a branch point of the catheter shaft, a merge point located near the most distal end of the catheter shaft, and a plurality of branch structures each including the plurality of electrodes and individually connecting the branch point and the merge point in a curved shape.

The non-deployed shape may be a petal shape formed by the plurality of branch structures, and the deployed shape may be a shape in which the petal shape is deployed in the axial direction.

Incidentally, an example of the rotation action of the near-distal end structure in response to the rotation operation is a torsional rotation action about the rotation axis.

According to the catheter according to one embodiment of the disclosure, since the operation mechanism is provided, when the near-distal end structure is rotated, the following effect is achieved. That is, the position of the electrode in the near-distal end structure can be freely adjusted in the radial direction or the circumferential direction about the axial direction while the length of the near-distal end structure in the axial direction is fixed. This makes it possible to improve the convenience when using the catheter.

### BRIEF DESCRIPTION OF DRAWINGS

FIG. 1A and FIG. 1B are schematic diagrams illustrating a schematic configuration example of a catheter according to an embodiment of the disclosure.

FIG. 2A and FIG. 2B are perspective views illustrating a schematic configuration example of a handle illustrated in FIGS. 1A and 1B.

FIG. 3 is a perspective view illustrating a schematic configuration example of a part of the handle illustrated in FIGS. 2A and 2B.

FIG. 4A, FIG. 4B and FIG. 4C are schematic diagrams illustrating an example of a deformed state near a distal end of the catheter shaft illustrated in FIGS. 1A and 1B.

FIG. 5A, FIG. 5B and FIG. 5C are schematic diagrams illustrating an example of another deformed state near the distal end of the catheter shaft illustrated in FIGS. 1A and 1B.

FIG. 6 is a schematic plan view illustrating a schematic configuration of a catheter according to a comparative example.

FIG. 7A and FIG. 7B are schematic diagrams illustrating an example of a rotation action near the distal end of the catheter shaft illustrated in FIGS. 1A and 1B.

FIG. 8A and FIG. 8B are schematic diagrams illustrating an example of another rotation action near the distal end of the catheter shaft illustrated in FIGS. 1A and 1B.

### DESCRIPTION OF EMBODIMENTS

Embodiments of the disclosure will be described below in detail with reference to the drawings. Note that the description will be given in the following order.
1. Embodiment (example of case of operation mechanism in which both a rotation operation and a slide operation are performed)
2. Modified Examples (examples of operation mechanism in which only the rotation operation is performed and the slide operation is not performed)

### 1. Embodiment

### A. Schematic Configuration

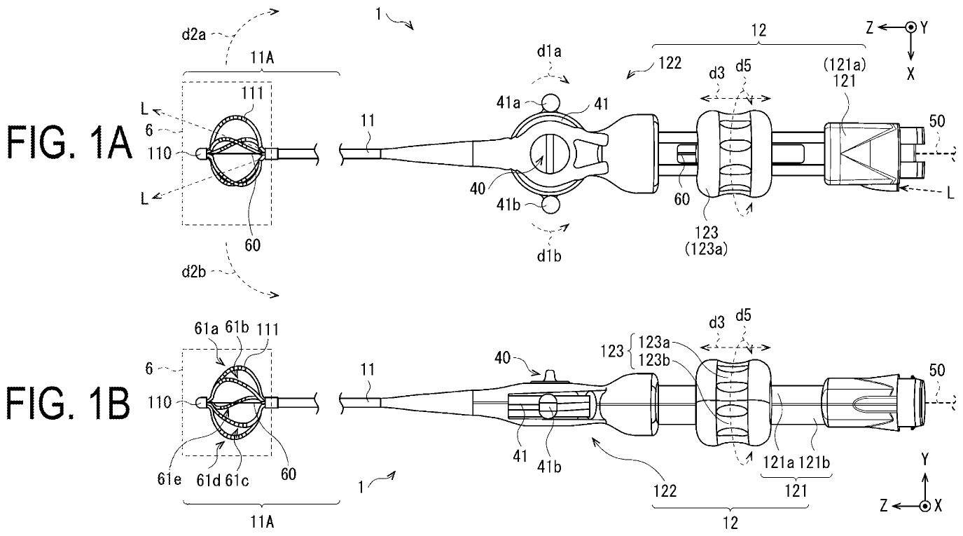

FIGS. 1A and 1B schematically illustrate a schematic configuration example of a catheter (electrode catheter 1) according to an embodiment of the disclosure. Specifically, FIG. 1A schematically illustrates a planar configuration example (Z-X plane configuration example) of the electrode catheter 1, and FIG. 1B schematically illustrates a side configuration example (Y-Z side configuration example) of the electrode catheter 1.

The electrode catheter 1 corresponds to a specific example of a “catheter” in the disclosure.

The electrode catheter 1 is a catheter that is inserted into a patient's body (e.g., inside the heart) through a blood vessel and is used for examination and treatment of arrhythmia or the like. Specifically, in the electrode catheter 1, measurement of electric potential near an affected area in the body, cauterization (ablation) of the affected area, and the like are performed using a plurality of electrodes (electrodes 111), which will be described later.

Further, as illustrated in FIGS. 1A and 1B, the electrode catheter 1 includes an irrigation mechanism that supplies (ejects) a predetermined irrigation liquid L (e.g., saline) from near a distal end (a near-distal end structure 6 described later) to the outside during such ablation.

As illustrated in FIGS. 1A and 1B, the electrode catheter 1 described above includes a catheter shaft 11 (catheter tube) serving as a catheter body (elongated portion) and a handle 12 mounted on a proximal end side of the catheter shaft 11.

The handle 12 corresponds to a specific example of a “handle” in the disclosure.

### Catheter Shaft 11

The catheter shaft 11 has a flexible tube-like structure (made of a hollow tube-like member) and has a shape that extends in an axial direction (Z-axis direction) of the catheter shaft 11 (see FIGS. 1A and 1B). Specifically, the length of the catheter shaft 11 in the axial direction is from several to several tens of times longer than the length of the handle 12 in the axial direction (Z-axis direction).

As illustrated in FIGS. 1A and 1B, the catheter shaft 11 includes a distal end portion (distal end flexible portion 11A) that is configured to have excellent relative flexibility. Further, as illustrated in FIGS. 1A and 1B, a predetermined near-distal end structure 6, which will be described later, is provided in the distal end flexible portion 11A. The catheter shaft 11 also has a so-called multi-lumen structure in which a plurality of lumens (inner holes, channels, through holes) are formed inside the catheter shaft 11 extending in the axial direction (Z-axis direction) of the catheter shaft 11. Various fine wires (lead wires 50, deflection wires, operation wires 60, or the like, which will be described later) are inserted through the lumen of the catheter shaft 11 while being electrically insulated from each other. Further, inside the catheter shaft 11, in addition to the lumens configured to receive insertion of these various fine wires, a lumen for supplying the above-described irrigation liquid L is formed extending in the axial direction.

The outer diameter of the catheter shaft 11 configured as such is, for example, from approximately 1.0 to 5.0 mm, and the length of the catheter shaft 11 in the axial direction is, for example, from approximately 300 to 1500 mm. Examples of the constituent material of the catheter shaft 11 include a thermoplastic resin, such as polyamide, polyether polyamide, polyurethane, polyether block amide (PEBAX (trade name)), and nylon.

As illustrated in FIGS. 1A and 1B, the near-distal end structure 6 includes a branch point (a position at the proximal end side of the near-distal end structure 6) of the catheter shaft 11, a merge point located near the most distal end (near a distal end tip 110 to be described later) of the catheter shaft 11, and a plurality of (five in this example) branch structures 61a to 61e that are portions that individually connect the branch point and the merge point in a curved shape. These branch structures 61a to 61e are spaced apart from each other at substantially equal intervals in a plane (X-Y plane) perpendicular to the axial direction (Z-axis direction) of the catheter shaft 11.

Further, as illustrated in FIGS. 1A and 1B, in these branch structures 61a to 61e, one or a plurality of electrodes 111 (four electrodes 111 in this example) are disposed spaced apart from each other at predetermined intervals in the curved extension directions of the branch structures 61a to 61e. Each of the electrodes 111 is a ring-shaped electrode. The distal end tip 110 is arranged at the merge point of the branch structures 61a to 61e (near the most distal end of the catheter shaft 11).

As described above, the electrodes 111 described above are, for example, electrodes for potential measurement or cauterization, and are made of a metal material with good electrical conductivity such as, aluminum (Al), copper (Cu), SUS, gold (Au), and platinum (Pt). The distal end tip 110 is constituted by a metal material similar to that of the electrodes 111, and is also constituted by a resin material such as a silicone rubber resin, polyurethane, or polycarbonate.

The electrodes 111 described above are individually electrically connected to the distal end sides of the lead wires 50. Further, the proximal end side of each lead wire 50 can be connected to the outside of the electrode catheter 1 from the inside of the handle 12 through the inside of the catheter shaft 11. Specifically, as illustrated in FIGS. 1A and 1B, the proximal end side of each lead wire 50 is taken out to the outside from the proximal end portion (connector portion) of the handle 12 in the Z-axis direction.

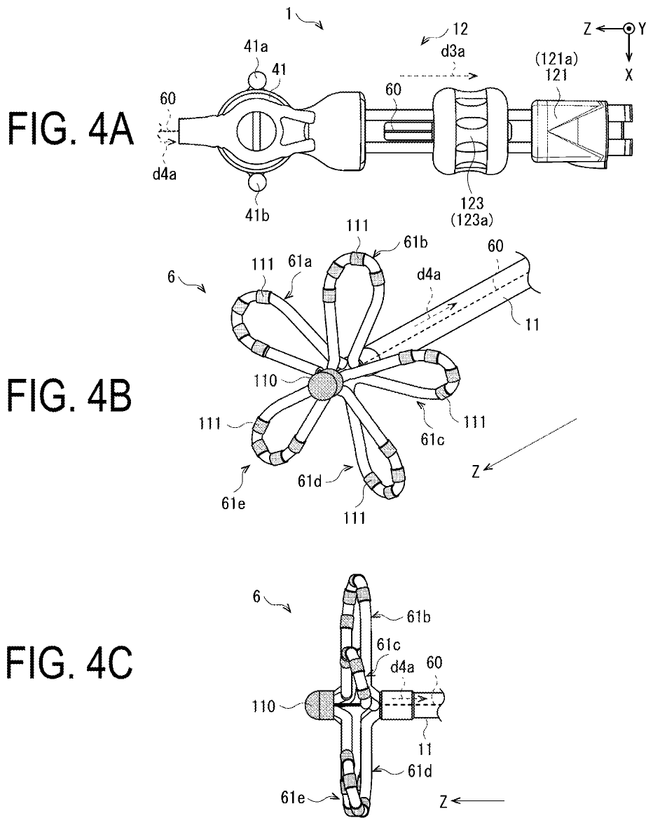

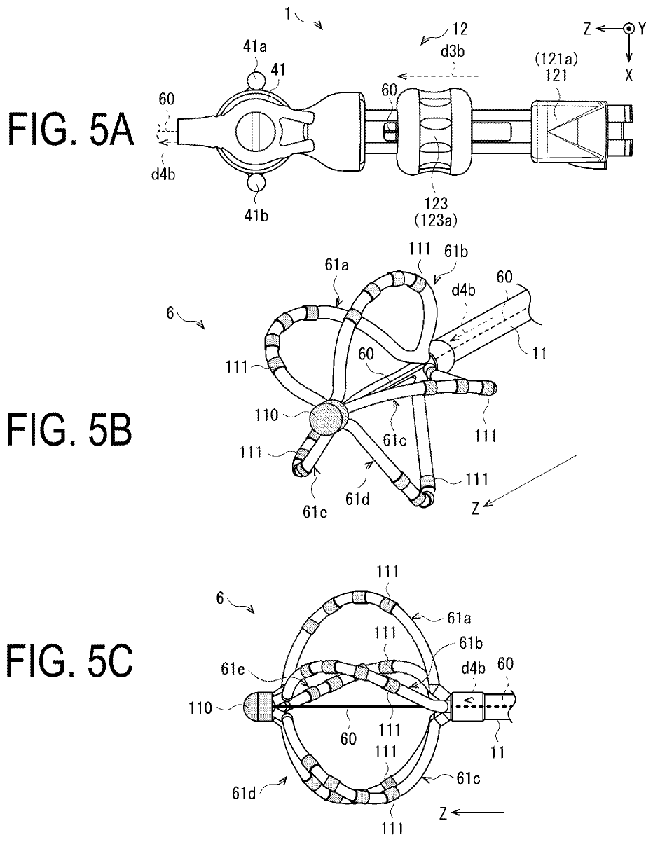

Here, the shape of the near-distal end structure 6 is configured to change (deform) in response to a slide operation on the handle 12 described later (a slide operation on an operation mechanism 123 to be described later). Specifically, the shape of the near-distal end structure 6 changes between a non-deployed shape (contracted shape: see FIGS. 4A to 4C to be described later) in which the near-distal end structure 6 is not deployed in the axial direction (Z-axis direction) and a deployed shape (expanded shape: see FIGS. 1A and 1B and FIGS. 5A to 5C to be described later) in which the near-distal end structure 6 is deployed from the non-deployed shape in the axial direction. Although details will be described later, an example of the non-deployed shape is a “petal shape” (example of a flat shape: see FIGS. 4A to 4C to be described later) formed by the plurality of branch structures 61a to 61e. On the other hand, an example of the deployed shape is a shape in which the petal shape (the branch structures 61a to 61e) is deployed in the axial direction (so-called “basket shape”: see FIGS. 1A and 1B and FIGS. 5A to 5C to be described later).

Note that the “basket shape” means that the shape formed by the plurality of the branch structures 61a to 61e resembles the curved pattern formed on the surface of a basketball, as illustrated in FIGS. 1A and 1B and FIGS. 5A to 5C, for example.

The non-deployed shape (and the petal shape) corresponds to a specific example of a “first shape” in the disclosure. Further, the deployed shape (and the basket shape) corresponds to a specific example of a “second shape” in the disclosure.

### Handle 12

The handle 12 is a portion that an operator (physician) grips (holds) when using the electrode catheter 1. As illustrated in FIGS. 1A and 1B, the handle 12 includes a handle body 121 mounted on the proximal end side of the catheter shaft 11 and a rotation operation portion 122.

The handle body 121 corresponds to a portion (gripping portion) that an operator actually grips, and has a shape extending in an axial direction (Z-axis direction) of the handle body 121. The handle body 121 is made of a synthetic resin such as, for example, polycarbonate, polyacetal, or an acrylonitrile-butadiene-styrene copolymer (ABS).

Although details will be described later, the rotation operation portion 122 is a portion that is operated during a deflection action for deflecting (bending) the vicinity (distal end flexible portion 11A) of the distal end of the catheter shaft 11 in both directions. The rotation operation portion 122 is used during this deflection action together with a pair of deflection wires (not illustrated). Specifically, during the deflection action, the rotation operation portion 122 is operated (rotated) by the operator of the electrode catheter 1. As illustrated in FIGS. 1A and 1B, the rotation operation portion 122 described above includes a lock mechanism 40 and a rotating plate 41.

Distal ends of the pair of deflection wires are fixed to the distal end side of the catheter shaft 11 (e.g., the proximal end side of the branch point in the near-distal end structure 6). Proximal end sides of the pair of deflection wires extend from the inside of the catheter shaft 11 to the inside of the handle 12 (inside of the handle body 121).

As illustrated in FIGS. 1A and 1B, the rotating plate 41 is a member attached to the handle body 121 such that the rotating plate 41 is rotatable about a rotation axis (Y-axis direction) perpendicular to the axial direction (Z-axis direction) of the rotating plate 41. The rotating plate 41 corresponds to a portion that is actually operated by the operator during the rotation operation, and has a substantially disk-like shape. Specifically, in this example, as indicated by arrows d1a and d1b in FIG. 1A, it is possible to perform an operation (rotation operation about the rotation axis in the Y-axis direction) of rotating the rotating plate 41 in both directions in the Z-X plane with respect to the handle body 121.

The lock mechanism 40 is a mechanism for fixing (locking) the rotational position of the rotating plate 41 within the Z-Y plane.

Here, as illustrated in FIGS. 1A and 1B, a pair of knobs 41a and 41b are provided integrally with the rotating plate 41 on a side surface of the rotating plate 41. In the example illustrated in FIGS. 1A and 1B, the knob 41a and the knob 41b are disposed at positions that are point-symmetrical about the rotation axis of the rotating plate 41. Each of these knobs 41a and 41b corresponds to a portion that is operated (pushed) by the fingers of one hand, for example, when the operator rotates the rotating plate 41. Note that the rotating plate 41 is constituted by a material (synthetic resin or the like) similar to that of the handle body 121 described above, for example.

A pair of fasteners (not illustrated) are provided on the rotating plate 41. These fasteners are members (wire fasteners) used for individually fixing the proximal ends of the pair of deflection wires by screwing or the like. Note that, with these fasteners, it is possible to freely adjust the retraction length near each proximal end when fixing the proximal ends of the pair of deflection wires.

### B. Detailed Configuration of Handle 12

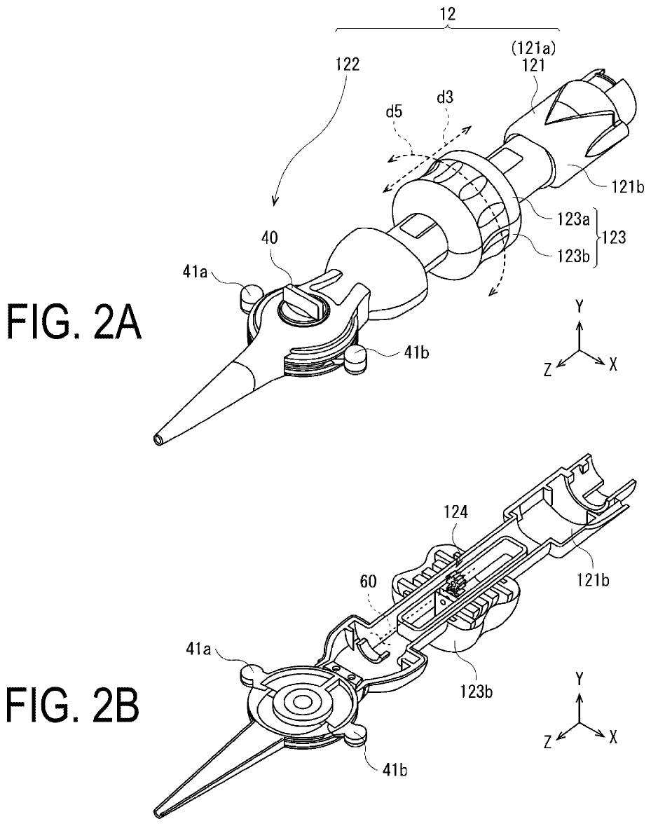

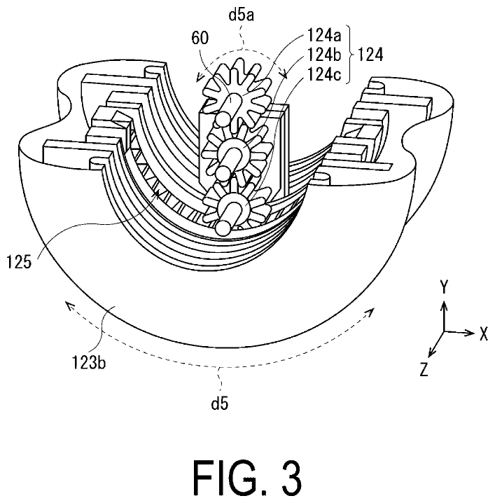

Next, with reference to FIGS. 2A, 2B and 3 in addition to FIGS. 1A and 1B, a specific configuration example of the handle 12 will be described.

FIGS. 2A and 2B are perspective views illustrating a schematic configuration example of the handle 12. Specifically, FIG. 2A is a perspective view illustrating a schematic configuration example of the handle 12, and FIG. 2B is an exploded perspective view illustrating a configuration example (a configuration example in which a handle member 121a to be described later is removed) of a portion of the handle 12. FIG. 3 is a perspective view illustrating a configuration example of a portion of the handle 12 illustrated in FIGS. 2A and 2B (a configuration example of portions of an operation member 123a and gears 124a to 124c to be described later).

First, as illustrated in FIGS. 2A and 2B, the handle body 121 is formed using a pair of handle members 121a and 121b that can be separated in the Y-axis direction. In other words, the handle body 121 is formed by linking these handle members 121a and 121b to each other.

In the handle body 121, a path through which the irrigation liquid L flows and a path through which the lead wire 50 passes may be arranged separately from each other. Specifically, the paths for the liquid L and the lead wire 50 are arranged separately from each other so as to be on opposite sides to each other with a gear 124 (see FIG. 2B) interposed therebetween.

Here, as illustrated in FIGS. 1A, 1B, 2A and 2B, the handle 12 (handle body 121) is provided with the operation mechanism 123 configured to be rotatable (see arrow d5) in both directions in the X-Y plane with respect to the handle body 121 about a rotation axis in the axial direction (Z-axis direction). The operation mechanism 123 is a portion that is rotationally operated by the operator (see arrow d5) when the near-distal end structure 6 is rotated about the rotation axis in the Z-axis direction while the axial length (length in the axial direction in the Z-axis direction) of the near-distal end structure 6 is fixed. Although details will be described later, the rotation action of the near-distal end structure 6 generated in response to this rotation operation is a torsional rotation action (helical rotation action) about the rotation axis in the Z-axis direction.

In the present embodiment, as illustrated in FIGS. 1A, 1B, 2A and 2B, the operation mechanism 123 is further configured to be slidable (see arrow d3) in the axial direction (Z-axis direction) in the handle body 121. During the deformation action in which the shape of the near-distal end structure 6 is changed between the non-deployed shape (petal shape) and the deployed shape (basket shape), the operator performs a bidirectional slide operation (see arrow d3) on the operation mechanism 123. As illustrated in FIGS. 1A, 2A and 2B, this slide operation on the operation mechanism 123 is performed in a rail (open portion) in the Z-axis direction formed on the handle body 121 (handle members 121a and 121b).

Further, the operation mechanism 123 can be set to any slide position in the axial direction (Z-axis direction) on the handle body 121. Thus, the shape of the near-distal end structure 6 during the deformation action can be set to any intermediate shape between the non-deployed shape (petal shape) and the deployed shape (basket shape) according to the slide position of the operation mechanism 123.

As illustrated in FIGS. 1A, 1B, 2A, 2B and 3, the operation mechanism 123 is configured using a pair of operation members 123a and 123b that can be separated in the Y-axis direction, similar to the handle body 121 described above. In other words, the operation mechanism 123 is formed by linking these operation members 123a and 123b to each other. As illustrated in FIGS. 2B and 3, a gear 124 is provided between the operation members 123a and 123b in the handle body 121. As illustrated in FIG. 3, the gear 124 is configured using three gears 124a, 124b, and 124c that are arranged side by side in the Y-axis direction and mesh with each other. As illustrated in FIG. 3, the gear 124c meshes with an internal gear 125 of the operation mechanism 123 (operation member 123b), and the gears 124a, 124b, and 124c mesh with each other. Thus, the following configuration is achieved. That is, each of the gears 124a, 124b, 124c is configured to rotate in conjunction with the above-described rotation operation of the operation mechanism 123 (see arrow d5a representatively illustrated in relation to the gear 124a in FIG. 3).

Here, the distal end side of the operation wire 60 (see FIGS. 1A, 2B, and 3) used during the rotation action and the deformation action of the near-distal end structure 6 is fixed to the near-distal end structure 6 (near the distal end tip 110). On the other hand, as illustrated in FIGS. 2B and 3, the proximal end side of the operation wire 60 is fixed to the operation mechanism 123 in the handle body 121 via the gear 124 described above. More specifically, in the example illustrated in FIG. 3, the proximal end side of the operation wire 60 is inserted into the gear 124a so that the proximal end side of the operation wire 60 is fixed to the operation mechanism 123 from the gear 124a and via the gears 124b and 124c.

### C. Action and Functions/Effects

Next, the action and functions/effects of the electrode catheter 1 of the present embodiment will be described in detail with comparison to a comparative example.

### C-1. Deflection Action of Distal End Flexible Portion 11A by Rotation Operation

First, in the electrode catheter 1, the shape of the catheter shaft 11 near the distal end (distal end flexible portion 11A) changes in both directions according to the rotation operation (the rotation operation about the rotation axis in the Y-axis direction described above) of the rotating plate 41 performed by the operator. That is, in measuring the electric potential near an affected area in the body or cauterizing the affected area as described above, the action of deflecting the distal end flexible portion 11A in both directions (the deflection action in both directions) is performed in response to the rotation operation described above.

Specifically, for example, when the operator grips the handle 12 (handle body 121) with one hand and operates the knob 41a with the fingers of that hand to rotate the rotating plate 41 in the direction of the arrow d1a (clockwise) in FIG. 1A, the following effect is achieved. That is, one deflection wire of the pair of deflection wires is pulled toward the proximal end side inside the catheter shaft 11. Then, the distal end flexible portion 11A of the catheter shaft 11 is curved (bent) in the direction indicated by the arrow d2a in FIG. 1A.

Further, for example, when the operator operates the knob 41b to rotate the rotating plate 41 in the direction of the arrow d1b (counterclockwise) in FIG. 1A, the following effect is achieved. That is, the other deflection wire of the pair of deflection wires is pulled toward the proximal end side inside the catheter shaft 11. Then, the distal end flexible portion 11A of the catheter shaft 11 is curved in the direction indicated by the arrow d2b in FIG. 1A.

In this manner, the operator can perform a (swing) deflection action in both directions in the catheter shaft 11 by rotating the rotating plate 41. By rotating the handle body 121 about its axis (within the X-Y plane), the bending direction (deflection direction) of the distal end flexible portion 11A of the catheter shaft 11 can be freely set in a state where the catheter shaft 11 is inserted into the patient's body, for example. In this manner, since the electrode catheter 1 is provided with a deflection mechanism for deflecting the distal end flexible portion 11A in both directions, the catheter shaft 11 can be inserted into the patient's body while changing shape near its distal end (distal end flexible portion 11A).

As described above, the potential measurement and cauterization (ablation) are performed at the distal end flexible portion 11A (the near-distal end structure 6 including the plurality of electrodes 111).

Further, in the present embodiment, the above-described irrigation liquid L is supplied to the electrode catheter 1 during the ablation. Specifically, for example, as illustrated in FIG. 1A, the liquid L is supplied into the handle body 121 from a side surface (liquid inlet) on the proximal end side of the handle body 121. Then, for example, as illustrated in FIG. 1A, the liquid L flows out (is ejected) to the outside from near the distal end of the electrode catheter 1 (near the above-described branch point in the near-distal end structure 6). This avoids damage caused by an excessive increase in the temperature of the procedure part during ablation and a thrombus sticking to the procedure part (blood retention is improved).

### C-2. Deformation Action of Near-Distal End Structure 6 by Slide Operation

Next, referring to FIGS. 4A to 5C, the deformation action of the near-distal end structure 6 of the catheter shaft 11 by the slide operation performed on the operation mechanism 123 will be described in detail.

FIG. 4 (FIGS. 4A to 4C) schematically illustrates an example of a deformed state (the state of the petal shape as an example of the non-deployed shape) near the distal end of the catheter shaft 11 (the near-distal end structure 6). FIG. 5 (FIGS. 5A to 5C) schematically illustrates an example of another deformed state (the state of the basket shape as an example of the deployed shape) near the distal end of the catheter shaft 11 (the near-distal end structure 6). Note that the deployed shape (basket shape) illustrated in FIGS. 5A to 5C is merely an example, and a shape obtained by slightly deflating (distorting) the shape illustrated in FIGS. 5A to 5C and the like may also be adopted, for example.

First, as indicated by the arrow d3a in FIG. 4A, for example, when the operation mechanism 123 slides toward the proximal end side of the handle body 121 by a slide operation performed on the operation mechanism 123 by the operator, the following effect is achieved. That is, as described above, the proximal end side of the operation wire 60 is fixed by the operation mechanism 123. In this case, for example, as indicated by the arrow d4a in FIGS. 4A to 4C, as the operation mechanism 123 slides toward the proximal end side, the operation wire 60 is also pulled toward the proximal end side. Then, as described above, since the distal end side of the operation wire 60 is fixed to the near-distal end structure 6 (near the distal end tip 110), for example, as illustrated in FIGS. 4B and 4C, the distal end tip 110 is pulled toward the proximal end side, and a shape in which the branch structures 61a to 61e are contracted toward the proximal end side is obtained. That is, the near-distal end structure 6 has the non-deployed shape (in this example, a shape substantially flattened in the X-Y plane). Specifically, in this example, as illustrated in FIG. 4B, the near-distal end structure 6 has the petal shape formed by the branch structures 61a to 61e.

On the other hand, as indicated by the arrow d3b in FIG. 5A, for example, when the operation mechanism 123 slides toward the distal end side of the handle body 121 by the slide operation performed on the operation mechanism 123 by the operator, the following effect is achieved. That is, in this case, as indicated by the arrow d4b in FIGS. 5A to 5C, for example, as the operation mechanism 123 slides toward the distal end side, the operation wire 60 is also pushed toward the distal end side. Then, for example, as illustrated in FIGS. 5B and 5C, the distal end tip 110 is pushed toward the distal end side, and a shape in which the branch structures 61a to 61e are deployed toward the distal end side is obtained. That is, the near-distal end structure 6 has the deployed shape (a shape in which the near-distal end structure 6 is deployed toward the distal end side in the Z-axis direction). Specifically, in this example, as illustrated in FIG. 5B, the near-distal end structure 6 has the basket shape formed by the respective branch structures 61a to 61e.

In this manner, the near-distal end structure 6 is deformed in response to the slide operation performed on the operation mechanism 123.

### C-3. Comparative Example

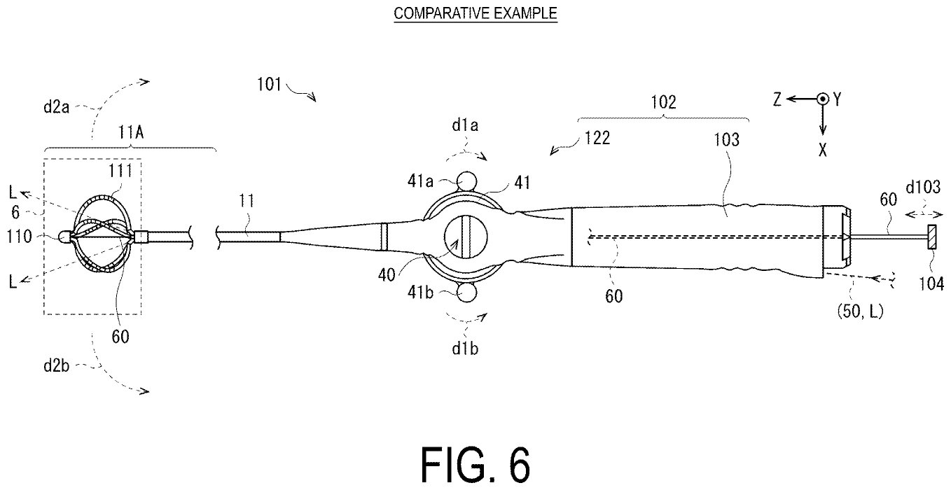

FIG. 6 is a schematic plan view (Z-X plan view) illustrating a schematic configuration of a catheter (electrode catheter 101) according to a comparative example.

The electrode catheter 101 of this comparative example includes a catheter shaft 11 having a near-distal end structure 6 and a handle 102 including a handle body 103 and a rotation operation portion 122. In other words, the electrode catheter 101 of this comparative example includes the handle 102 and the handle body 103 instead of the handle 12 and the handle body 121 in the electrode catheter 1 of the present embodiment (see FIGS. 1A and 1B).

Specifically, as illustrated in FIG. 6, in this handle body 103, a push-in operation portion 104 is provided instead of the operation mechanism 123 (a member on which the slide operation is performed) in the embodiment described above. The proximal end side of the operation wire taken out from the proximal end of the handle body 103 is attached to the push-in operation portion 104. When the operator operates the push-in operation portion 104 in the direction of an arrow d103 (Z-axis direction: the extension direction of the operation wire 60), an operation of pushing the operation wire 60 into the handle body 121 is performed. As a result, the near-distal end structure 6 is deformed in the same manner as in the present embodiment. That is, in this comparative example, the operation in the direction of the arrow d103 performed on the push-in operation portion 104 corresponds to the operation for deforming the near-distal end structure 6.

In the handle body 103 of this comparative example, unlike the handle body 121 of the present embodiment illustrated in FIGS. 1A and 1B, the irrigation liquid L is introduced from the side surface of the handle body 103 on the proximal end side, and the lead wire 50 is also pulled out. That is, since the operation wire 60 is pulled out from the proximal end of the handle body 103, unlike the present embodiment, the lead wire 50 is pulled out from the side surface rather than from the proximal end of the handle body 103.

In this comparative example, in a state where the operator grips the handle body 103 with one hand, the above-described operation (operation for deforming the near-distal end structure 6) performed on the push-in operation portion 104 is performed using the other hand of the operator. Thus, since both hands of the operator are required when performing the operation on the push-in operation portion 104, the operation for deforming the near-distal end structure 6 becomes complicated (it is difficult to easily perform the operation for deformation).

Accordingly, the convenience of using the electrode catheter 101 of the comparative example is impaired.

### C-4. Rotation Action of Near-Distal End Structure 6 by Rotation Operation

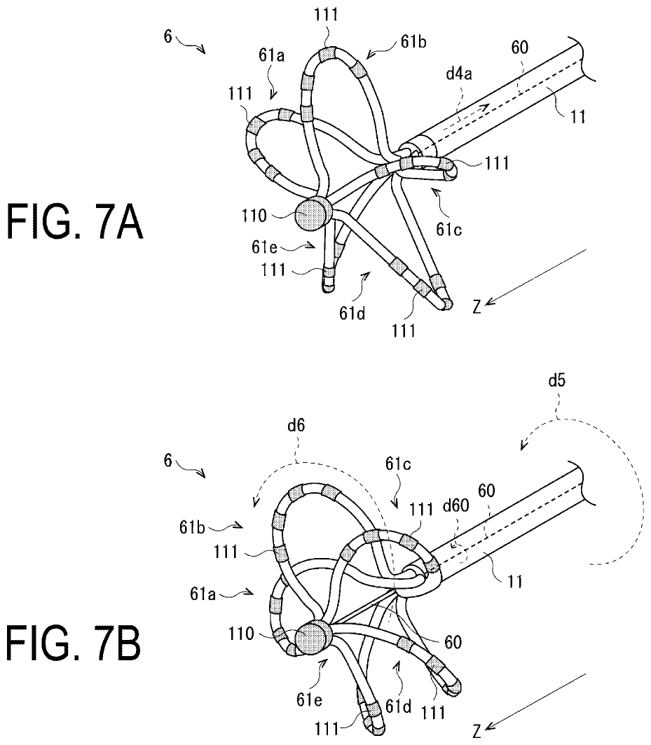

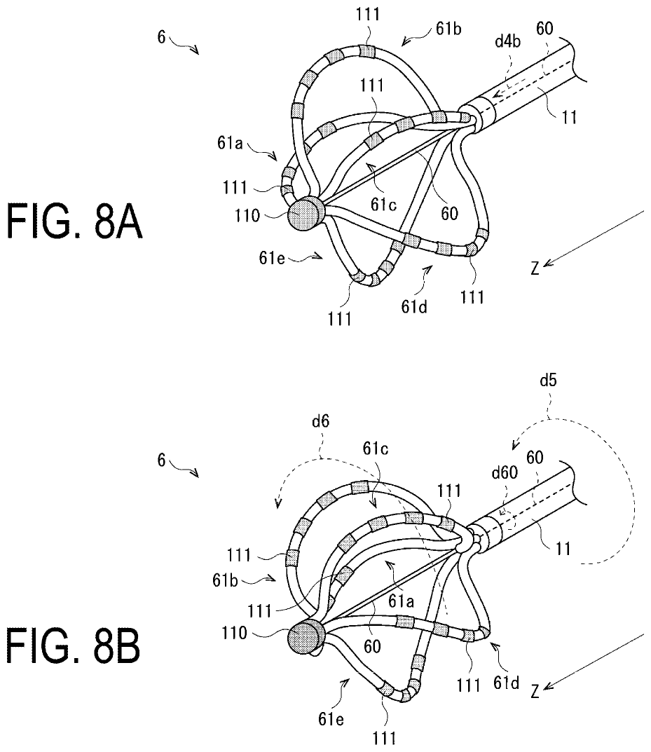

Next, referring to FIGS. 7A to 8B, the rotation action (torsional rotation action) of the near-distal end structure 6 of the catheter shaft 11 by the rotation operation (rotation operation about the rotation axis in the Z-axis direction described above) performed on the operation mechanism 123 will be described in detail.

FIG. 7 (FIGS. 7A and 7B) is a schematic diagram illustrating an example of a rotation action near the distal end of the catheter shaft 11 (the near-distal end structure 6). FIG. 8 (FIGS. 8A and 8B) is a schematic diagram illustrating another example of a rotation action near the distal end of the catheter shaft 11 (the near-distal end structure 6). Specifically, FIGS. 7A and 7B illustrate an example of the rotation action in the case of the state of the petal shape as an example of the above-described non-deployed shape. FIGS. 8A and 8B illustrate an example of the rotation action in the case of the state of the basket shape as an example of the above-described deployed shape.

First, in the case where the near-distal end structure 6 is set to the non-deployed shape (petal shape) as illustrated in FIG. 7A, when a rotation operation (rotation operation about the rotation axis in the Z-axis direction) is performed on the operation mechanism 123 by the operator as indicated by the arrow d5 in FIG. 7B, for example, the following effect is achieved. That is, as described above, since the proximal end side of the operation wire 60 is fixed by the operation mechanism 123, the operation wire 60 also rotates about the rotation axis in the Z-axis direction in response to the rotation operation on the operation mechanism 123 (see arrow d60 in FIG. 7B). Then, as described above, since the distal end side of the operation wire 60 is fixed to the near-distal end structure 6 (near the distal end tip 110), the distal end tip 110 is also rotated about the rotation axis in the Z-axis direction. As a result, each of the branch structures 61a to 61e and each of the electrodes 111 in the near-distal end structure 6 is subjected to a rotation action (torsional rotation action) about the rotation axis in the Z-axis direction, as indicated by the arrow d6 in FIG. 7B, for example.

On the other hand, in the case where the near-distal end structure 6 is set to the deployed shape (basket shape) as illustrated in FIG. 8A, for example, when the operator performs a rotation operation on the operation mechanism 123 as indicated by the arrow d5 in FIG. 8B, the following effect is achieved. That is, also in this case, as in the case of FIGS. 7A and 7B described above, the operation wire 60 is also rotated about the rotation axis in the Z-axis direction in response to the rotation operation performed on the operation mechanism 123 (see arrow d60 in FIG. 8B). Then, as in the case of FIGS. 7A and 7B described above, the distal end tip 110 of the near-distal end structure 6 is also rotated about the rotation axis in the Z-axis direction. As a result, each of the branch structures 61a to 61e and each of the electrodes 111 in the near-distal end structure 6 are subjected to a rotation action (torsional rotation action) about the rotation axis in the Z-axis direction, as indicated by the arrow d6 in FIG. 8B, for example.

In this manner, rotation action (torsional rotation action) of the near-distal end structure 6 is achieved in response to the rotation operation performed on the operation mechanism 123.

### C-5. Functions/Effects of Present Embodiment

As described above, in the electrode catheter 1 of the present embodiment, the following functions/effects, for example, are obtained with the configuration described above.

First, the electrode catheter 1 of the present embodiment includes the operation mechanism 123 that is configured to be rotatable with respect to the handle body 121 about the rotation axis in the Z-axis direction described above and is rotationally operated when the near-distal end structure 6 is rotationally operated. Accordingly, the near-distal end structure 6 rotates about the above-described rotation axis in response to the rotation operation, and the following effect is achieved. That is, the position of each electrode 111 in the near-distal end structure 6 can be freely adjusted in the radial direction or the circumferential direction about the axial direction (Z-axis direction) while the axial length in the Z-axis direction of the near-distal end structure 6 is fixed. Therefore, the convenience of using the electrode catheter 1 can be improved in the present embodiment.

In the present embodiment, the operation mechanism 123 described above is further configured to be slidable in the handle body 121 in the axial direction (Z-axis direction). During the deformation action in which the shape of the near-distal end structure 6 is changed between the non-deployed shape (petal shape) and the deployed shape (basket shape), a slide operation in the Z-axis direction is performed on the operation mechanism 123.

As a result, in the present embodiment, during this deformation action, in a state where the operator holds the handle body 121 with one hand, the operator can perform the operation on the operation mechanism 123 with that hand (the same hand). That is, for example, as in the comparative example described above, an operation using both hands of the operator as in the case of an operation of pushing the operation wire 60 against the handle body 121 using the other hand is not required, and the operation (the slide operation) during the deformation action can be easily performed using only one hand of the operator.

In addition, since the position in the radial direction of each electrode 111 in the near-distal end structure 6 can also be adjusted in accordance with the deformation action, for example, the position of each electrode 111 can also be adjusted in accordance with the thickness (size of the diameter) of the blood vessel of the patient. Specifically, countermeasures can be taken such that, for example, the deployed shape (basket shape) described above is set for a patient having a thin blood vessel (with a small diameter) and the non-deployed shape (petal shape) described above is set for a patient having a thick blood vessel (with a large diameter).

Accordingly, the convenience of using the electrode catheter 1 can be further improved in the present embodiment.

Further, since the slide operation is performed using the operation mechanism 123, the following effects can be obtained, for example. That is, for example, with the handle 12 placed on a predetermined table, it is possible to easily perform the above-described rotation operation with one hand while performing the slide operation with the other hand. Further, unlike the handle body 103 in the comparative example, the lead wire 50 can be easily pulled out from the proximal end of the handle body 121 while being separated from the inflow path of the irrigation liquid L.

Further, in the present embodiment, since the shape of the near-distal end structure 6 can be set to any intermediate shape between the non-deployed shape and the deployed shape according to the slide position of the operation mechanism 123 in the handle body 121, the following effect is achieved. That is, the convenience when using the electrode catheter 1 can be further improved.

In addition, in the present embodiment, the distal end side of the operation wire 60 used in the above-described rotation action and deformation action is fixed to the near-distal end structure 6 (near the distal end tip 110 described above). The proximal end side of the operation wire 60 is fixed to the operation mechanism 123 in the handle body 121 via the gear 124 (gears 124a to 124c) that rotates in conjunction with the rotation operation. Accordingly, since the rotation action and the deformation action can be easily performed, convenience when using the electrode catheter 1 can be further improved.

### 2. Modified Examples

The disclosure is described above with reference to the embodiment, but the disclosure is not limited to the embodiment, and various modifications can be made.

For example, the shape, arrangement position, size, number, material, and the like of the components described in the above-described embodiment are not limited, and other shapes, arrangement positions, sizes, numbers, materials, and the like may be used.

Specifically, for example, in the above-described embodiment, a specific configuration of the catheter shaft 11 is described as an example, but the catheter shaft 11 need not include all of the above-described members and may further include other members. Specifically, for example, a leaf spring that can be deformed in the deflection direction may be provided as a swing member inside the catheter shaft 11. Further, for example, the arrangement, shape, and number of (one or a plurality of) the electrodes 111 near the distal end of the catheter shaft 11 (in the near-distal end structure 6) are not limited to those described in the above-described embodiment. In addition, the shapes of the near-distal end structure 6 (the non-deployed shape and the deployed shape) are not limited to the shapes described in the embodiment (the petal shape, the basket shape, and the like as an example of the flat shape) and may be other non-deployed shapes or other deployed shapes. Furthermore, the configuration of the near-distal end structure 6 itself (e.g., the arrangement, shape, and number of the branch points, the merge points, and the plurality of branch structures described above) is not limited to the configuration described in the above-described embodiment, and may be another configuration.

Further, in the above-described embodiment, a specific configuration of the handle 12 (the handle body 121, the rotation operation portion 122, the operation mechanism 123, and the like) is described as an example, but the handle 12 need not include all of the above-described members and may further include other members. Specifically, for example, the type of shape when the near-distal end structure 6 is deformed is not limited to the case where the shape can be set to any intermediate shape as described in the embodiment and may be set to another case. That is, for example, the shape may be settable to only a plurality of types of preset intermediate shapes instead of any intermediate shape. Alternatively, for example, the shape may be set to only two types of shapes, that is, the non-deployed shape and the deployed shape (cannot be set to an intermediate shape). In the above-described embodiment, the case where the rotation action of the near-distal end structure 6 in response to the rotation operation on the operation mechanism 123 is the torsional rotation action about the rotation axis is described as an example, but the disclosure is not limited to this example. That is, the rotation action of the near-distal end structure 6 may be another rotation action other than such a torsional rotation action. Further, in the above-described embodiment, the case of the operation mechanism 123 subjected to both the rotation operation and the slide operation described above is described as an example, but the disclosure is not limited to this example. That is, for example, in some cases, the operation mechanism may be configured such that only the above-described rotation operation is performed and the above-described slide operation is not performed.

In addition, the shape near the distal end of the catheter shaft 11 is not limited to that described in the embodiment. Specifically, in the embodiment, the electrode catheter 1 is described as a type (bi-direction type) in which the shape near the distal end of the catheter shaft 11 changes in two directions in response to the rotation operation on the rotating plate 41 as an example. However, the type of electrode catheter 1 is not limited to this. That is, for example, the electrode catheter may be of a type (single-direction type) in which the shape near the distal end of the catheter shaft 11 changes in one direction in response to the rotation operation on the rotating plate 41. In this case, only one deflection wire is provided.

In addition, in the embodiment, the electrode catheter 1 that ejects the irrigation liquid L to the outside (having an irrigation mechanism) is described as an example. However, the disclosure is not limited to this example, and may be applied to, for example, an electrode catheter that does not have such an irrigation mechanism. Further, in the embodiment, the electrode catheter 1 that performs the above-described potential measurement and cauterization (ablation) is described as an example. However, the disclosure is not limited to this example, and may be applied to, for example, an electrode catheter used for other applications.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A catheter comprising:
a catheter shaft extending in an axial direction and having a near-distal end structure including a plurality of electrodes; and
a handle attached to a proximal end side of the catheter shaft,
the handle including:
a handle body extending in the axial direction; and
an operation mechanism configured to be rotatable with respect to the handle body about a rotation axis extending in the axial direction, the operation mechanism being rotationally operated when the near-distal end structure is rotationally operated about the rotation axis while a length of the near-distal end structure in the axial direction is fixed.

2. The catheter according to claim 1, wherein
the operation mechanism is further configured to be slidable in the axial direction in the handle body and is slid in the axial direction during a deformation action in which a shape of the near-distal end structure is changed between a first shape and a second shape,
the first shape is a non-deployed shape in which the near-distal end structure is not deployed in the axial direction, and
the second shape is a deployed shape in which the near-distal end structure is deployed from the non-deployed shape in the axial direction.

3. The catheter according to claim 2, wherein
the near-distal end structure is settable to any intermediate shape between the non-deployed shape and the deployed shape according to a slide position of the operation mechanism in the handle body.

4. The catheter according to claim 2, wherein
a distal end side of an operation wire used in the rotation action and the deformation action is fixed to the near-distal end structure, and
a proximal end side of the operation wire is fixed to the operation mechanism in the handle body via a gear configured to rotate in conjunction with the rotation operation.

5. The catheter according to claim 2, wherein
the near-distal end structure includes:
a branch point of the catheter shaft;
a merge point located near a most distal end of the catheter shaft; and
a plurality of branch structures respectively including the plurality of electrodes, the plurality of branch structures individually connecting the branch point and the merge point in a curved shape,
the non-deployed shape is a petal shape formed by the plurality of branch structures, and
the deployed shape is a shape in which the petal shape is deployed in the axial direction.

6. The catheter according to claim 1, wherein
a rotation action of the near-distal end structure in response to the rotation operation is a torsional rotation action about the rotation axis.
